(19) United States
(12) Patent Application Publication
(10) Pub. No.: US 2025/0129718 A1
(43) Pub. Date: Apr. 24, 2025

(54) METHOD FOR DETERMINING RESIDUAL OIL SATURATION BY MISCIBLE GAS INJECTION (SORM)

(71) Applicant: PETRÓLEO BRASILEIRO S.A. – PETROBRAS, Rio de Janeiro (BR)

(72) Inventors: Raphael Augusto MELLO VIEIRA, Rio de Janeiro (BR); Lucas Pinto TORRES DO NASCIMENTO, Rio de Janeiro (BR); Daniel Marcos PIRES TEIXEIRA DE SOUZA, Rio de Janeiro (BR); Samuel Da Silva FERREIRA DOS SANTOS, Rio de Janeiro (BR)

(73) Assignee: PETRÓLEO BRASILEIRO S.A. – PETROBRAS, Rio de Janeiro, RJ (BR)

(21) Appl. No.: 18/919,175

(22) Filed: Oct. 17, 2024

(30) Foreign Application Priority Data
Oct. 24, 2023 (BR) 1020230221491

(51) Int. Cl. E21B 49/08; E21B 47/11; E21B 49/00

(52) U.S. Cl. CPC E21B 49/0875; E21B 47/11; E21B 49/008

(57) ABSTRACT

The present invention relates to a method for determining residual oil saturation by miscible gas injection (Sorm), wherein the method comprises a first volumetric balance step, followed by a partitioning tracer injection step. Both steps are performed sequentially and independently in a closed system.

### FIELD OF THE INVENTION

The present invention relates to a method for determining residual oil saturation by miscible gas injection (Sorm), wherein the method comprises a first volumetric balance step, followed by a partitioning tracer injection step. Both steps are performed in situ in a sequential and independent manner.

The invention is applied in the area of reservoir engineering, in fields subjected to miscible gas injection with the following objectives: secondary oil recovery; advanced oil recovery or carbon capture, use and storage (CCUS). Thus, the area of application of the method of the present invention is in the modeling, simulation and evaluation of reservoirs as well as in oil recovery technologies.

### GROUNDS OF THE INVENTION

Most of the fields subjected to gas injection, whether rich or poor in CO₂, do so in a so-called miscible condition, where the interfacial tension between the gas and the oil is zero.

Although more efficient than immiscible gas injection, miscible injection cannot remove all the oil from the reservoir rocks, leaving a fraction of oil that remains trapped in the reservoir. The remaining part is not produced due to sweeping deficiencies, even on a microscopic scale, and is composed of the heaviest and most viscous fractions of the original hydrocarbon mixture. This composition is due to the intense mass transfer effect between the gaseous and oily phases after the passage of the miscible front, which preferentially extracts the light components and leaves the heavy ones.

This fraction of oil that remains trapped in the reservoir is called residual oil saturation (Ros or Sor from Portuguese), which is an important parameter for characterizing reservoirs because it is an important input for reserve calculations and mathematical reservoir simulations that predict oil production curves, thus impacting the valuation of the deposits and the economic attractiveness of exploration projects.

This parameter can be determined using different techniques and at different scales, including direct measurements of fluid saturation in core samples (e.g. retort or Dean Stark methods); laboratory tests of fluid injection in core samples (e.g. relative permeability tests); well logging; injection of tracers in wells (e.g. Single-Well Tracer Test-SWTT); and mathematical simulations in digital rock twins (e.g. Digital Rock).

Regarding laboratory methods of fluid injection, the industry standard is to determine residual oil saturation in immiscible conditions. In rare cases, measurements are made in conditions close to miscible. Measurements in fully miscible conditions are even scarcer.

In cases where it is necessary to perform gas injection in predominantly miscible conditions, the residual oil saturation values obtained by traditional methods are, as a rule, much higher than the real ones, which can lead to underestimation of reserves and oil production curves. In this sense, it is worth noting that obtaining the residual oil saturation value in miscible gas injections (Sorm) is much rarer given the difficulty of doing so under exact and representative conditions of temperature and pressure (pore pressure and subsurface overburden pressure) found in the reservoir rocks.

The scenario found in traditional methods of measuring residual oil saturation under conditions of immiscibility between the oil and the injected phase, such as in conventional water or gas injections, is also conditioned by the fact that immiscibility is a condition that facilitates determinations of relative permeability, a test generally associated with laboratory Sor measurements. In this scenario, the existence of both phases throughout the displacement process is guaranteed and the average saturations of the fluids inside the core plug can be determined by simple volumetric balance, regardless of the flow regime. In the miscible scenario, there may be times when there is single-phase flow in a certain portion of the rock, in addition to intense mass transfers between the displacing and displaced phases, with consequent variations in saturation. In this way, volumetric balance throughout the injection becomes operationally impossible.

However, when the objective becomes solely to measure Sor at the end of gas injection, it is possible to use specific volumetric balance methodologies that allow this value to be obtained. However, depends this calculation on some calibrations and data reconciliations that can make it uncertain, such as the need to know parameters such as the oil formation volume factor (Bo); volume of condensate in the injected gas and dead volume at the outlet of the high-pressure lines, downstream of the core sample.

Therefore, it is desirable to have a second methodology to determine in situ oil saturation, which is functional in reservoir conditions and can be applied immediately after the end of gas injection, without the need to depressurize and remove the sample from the sample holder, since its depressurization would already cause a change in Sorm, due to the release and expansion of gas.

From this perspective, tracer injection is a very accurate technique, in which small quantities of two tracer chemicals are simultaneously injected, dissolved in water. One of them has an affinity only for the aqueous phase (non-partitioning tracer) and the other also has an affinity for the oily phase (partitioning tracer). As the water containing the tracers flows and comes into contact with the residual, immobile oil, the molecules of the partitioning tracer dissolve (partition) in the oily phase and also remain immobile during the period of time they remain there. The process is dynamic and, when they return to the aqueous phase, they begin to migrate again along with this fluid. The non-partitioning tracer, in its turn, remains in the aqueous phase at all times and, therefore, always flows at the same speed as the water. The net result of this process is a separation of the production profiles of the partitioning and non-partitioning tracers, with the partitioning tracer suffering a delay in its production.

Therefore, the great advantage of the tracer injection technique is that Sor depends only on the degree of separation between the tracer profiles and the water-oil partition coefficient of the partitioning tracer, which is also easily measured in the laboratory.

Thus, the objective of the present invention was to develop and implement a method that would allow the quantification of Sor with less uncertainty. This was possible thanks to the sequential in situ application of two distinct and independent techniques that could be validated against each other: volumetric balance and partitioning tracer injection. The sequential in situ combination of the two techniques reduces the uncertainty regarding the quantification of Sor. Thus, the more accurate determination of Sor values results in improved predictability of production curves and reserves of oil fields, as well as in the development of optimized exploration projects.

### BACKGROUND OF THE INVENTION

The state of the art discloses the isolated application of volumetric balance and partitioning tracer injection techniques to determine saturation in residual oil, with volumetric balance being the most common technique. However, these techniques are performed alone, i.e., it is not a sequential method without the need to remove the sample from the holder or sample holder. Consequently, removing the sample from the equipment results in depressurization and consequent gas evolution, thus causing a decrease in the volume of oil due to the release of gas from the residual oil. This phenomenon changes the saturation of the residual oil and, consequently, increases the uncertainty about the final result.

In situ monitoring methods known in the state of the art are based on physical techniques, such as X-ray or Nuclear Magnetic Resonance. However, these methods are often not fast and accurate enough to monitor such saturation variations, especially in miscible recovery processes. It is also common to add chemical substances to the fluids in order to ensure image contrast/attenuation between phases. One drawback of this is that such substances can change the properties of the fluids and interfere with the behavior to be assessed (for example, miscibility).

The method of the present invention ensures reliable quantification of residual oil saturation, since the volumetric balance and partitioning tracer injection steps occur sequentially without the need to remove the sample from the system, thus avoiding gas evolution. Therefore, due to the sequential in situ steps, the method of the present invention has clear advantages over the state of the art, reducing the uncertainty in the results obtained due to the interference of external factors in open systems.

### SUMMARY OF THE INVENTION

The objective of the present invention is to determine the residual oil saturation at gas miscible injection (Sorm) in rock samples and reservoir conditions close to real ones, through two distinct and independent techniques applied in sequence (volumetric balance and partitioning tracer injection) in situ. The Sorm values obtained are much closer to the real ones (and significantly lower) than those obtained by existing traditional methods.

The method according to the present invention obtains two values for the same parameter, through distinct techniques performed sequentially in a closed system, consequently reducing the uncertainty of the parameter obtained.

### BRIEF DESCRIPTION OF THE FIGURES

In order to make the invention easier to understand, the Figures numbered 1 to 8, which accompany this specification and are an integral part thereof, are indicated by way of illustration, but without the intention of limiting the invention.

FIG. 1 illustrates the system used in the present invention.

FIG. 2 illustrates the adjustments of the partitioning (ethyl acetate) and non-partitioning (ethanol) tracer profiles for one of the samples evaluated (Rock 5).

FIG. 3 illustrates the schematic representation of the several portions of a reservoir subjected to miscible gas injection and their residual oil saturations.

FIG. 4 illustrates the behavior of oil saturation under different injection conditions for one of the samples evaluated (Rock 4).

FIG. 5 illustrates the behavior of residual oil saturation as a function of the CO₂ content in the injected gas for one of the samples evaluated (Rock 1).

FIG. 6 illustrates the behavior of oil saturation as a function of the continuous and alternating water and gas injection mode (Continuous×WAG (Water-Alternating-Gas)) for one of the samples evaluated (Rock 1).

FIG. 7 illustrates the behavior of oil saturation as a function of the gas injection mode (Continuous×WAG) for one of the samples evaluated (Rock 10).

FIG. 8 illustrates the behavior of oil saturation as a function of the gas injection mode (Continuous×WAG) for one of the samples evaluated (Rock 13).

### DETAILED DESCRIPTION OF THE INVENTION

The present invention relates to a method for determining residual oil saturation by miscible gas injection (Sorm), in which the method comprises a first volumetric balance step, followed by a partitioning tracer injection step, both steps being performed in situ through a closed system.

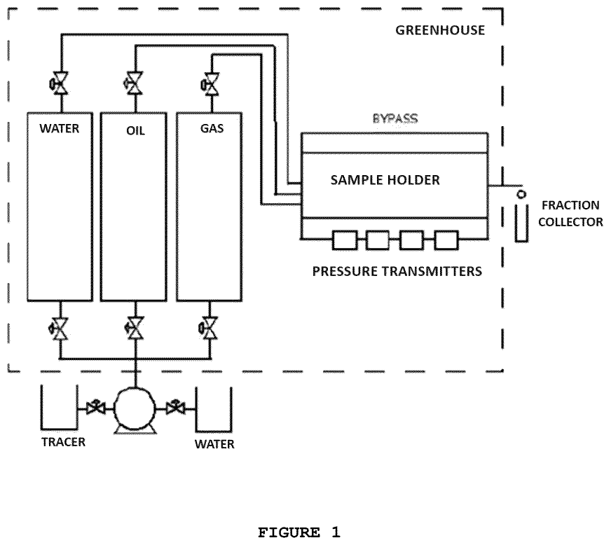

The system used in the present application (FIG. 1) focuses mainly on performing fluid displacement tests in porous media in reservoir conditions, fully reproducing the temperature; pore pressure and subsurface overburden pressure (close to the real reservoir condition). The system is composed of a large oven; a sample holder for 1.5″ diameter plugs; a sophisticated set of pumps; a three-phase separator; fluid accumulator vessels; electropneumatic valves; piping; pressure and temperature sensors and remote monitoring and control equipment. The system is suitable for acid service, which makes it suitable for working with fluids containing CO₂.

In situ method for determining residual oil saturation by miscible gas injection comprises the sequential steps of:
(a) volumetric balance; wherein the volumetric balance step comprises:
   (a₁) filling the sample holder bypass and transducer lines with deaerated water;
   (a₂) injecting live oil through the sample;
   (a₃) filling the gas pumps with the test gas;
   (a₄) injecting miscible gas with collection of the oil produced under atmospheric conditions (dead oil) over time, at a flow rate of the order of 0.5 to 1 cm³/min;
   (a₅) determining the volume of condensate produced by the gas;
   (a₆) determining the dead volume at the sample holder outlet through successive injections of dead water (deionized water) through the sample holder bypass and gas through the sample; and
   (a₇) calculating the determination of Sorm;
(b) injecting partitioning tracer; wherein the partitioning tracer injection step comprises:
   (b₁) injecting deaerated water through the sample, after completing the gas injection of step (a₄) of volumetric balance;
   (b₂) injecting an aqueous solution containing 1% v/v of partitioning tracer and 1% v/v of non-partitioning tracer at a flow rate of 1 cm³/min, collecting samples under atmospheric conditions at each fraction of 5% of the porous volume;
   (b₃) forwarding of the collected samples for quantification of tracers, by means of gas chromatography and/or spectrophotometry in the infrared region; and
   (b₄) calculating the determination of Sorm.

The objective of step (a₁) is to ensure that all the oil produced during the gas injection comes solely from the rock sample.

The objectives of step (a₂) are to fill the outlet dead volume with oil; and determine the “Live Oil Formation Volume Factor (Bo)”. Since the quantification of residual oil saturation upon injection of miscible gas must be done by collecting dead oil produced outside the system, under atmospheric conditions, it is necessary to obtain a calibration of the dead oil volume produced under atmospheric conditions for the same live oil volume injected under reservoir conditions. This calibration factor is known as the “Live Oil Formation Volume Factor (Bo)”. The injection of live oil must be stopped after the values of dead oil volume produced per live oil volume injected have stabilized.

Regarding step (a₅) of determining the condensate volume, it is worth noting that at the end of the injection of some porous volumes of gas, the production of oil from the sample ceases, possibly leaving the production of condensate, which is the light components that were dissolved in the gas under reservoir conditions and condense upon depressurization and cooling to atmospheric conditions. The condensate volume produced under ambient conditions for each volume of gas injected under reservoir conditions must be known so that it can be subsequently discounted from the observed oil production. The drier (poor in condensable light components) the gas, the smaller this condensate volume will be and the less relevant the correction will be.

The determination of the dead volume in step (as) can optionally occur through other methods, such as the expansion of inert gas or successive injections of water and oil.

The injection of deionized water in step (b₁) is sometimes important to avoid damage to the equipment that quantifies the tracers, which could be caused by the presence of dissolved salts.

In the injection of tracers in step (b₂), with a view to determining residual oil saturation, two substances are injected simultaneously, one with affinity only for the aqueous phase (non-partitioning tracer) and the other with affinity for both the aqueous and oil phases (partitioning tracer). The degree of lag (delay) of the elution profile of the partitioning tracer in relation to the elution profile of the non-partitioning tracer is directly proportional to the residual oil saturation. The partitioning tracer used was ethyl acetate and the non-partitioning tracer was ethanol.

The use of this technology allows the determination of residual oil saturation in situ; without the need to depressurize, deconfine and remove the sample from the sample holder. In general, the measurement accuracy level is approximately ±2 percentage points of saturation. This is an alternative/complementary method for quantifying residual oil saturation by volumetric balance, helping to increase the level of certainty regarding the final value of the determined Sorm, especially in miscible gas injections, where residual oil values are generally very low.

The Sorm calculations by volumetric balance (step a₇) and by injection of tracers (step b₄) are performed as follows:

### Determination of the Water-Oil Partition Coefficient

Before the Sorm calculations by volumetric balance and by injection of partitioning tracer, the partition coefficient of the partitioning tracer is determined. This coefficient is essential information for determining, analytically or numerically, the residual oil saturation using tracer injection technology. It is therefore important that the measurement of this parameter is made with the water and oil actually used, as well as at the temperature of the displacement test. The state of the art shows that it is indifferent whether the measurement is made with live or dead oil or even at a pressure equivalent to that used in the experiment.

The partition in the oil causes the partitioning tracer to lag behind the non-partitioning tracer, since when it is in the residual (immobile) oil phase it does not flow at the same speed as the water that carries the non-partitioning tracer.

The partition coefficient is calculated using Equation 1 below.

$$K d = \frac{Co}{Ca} = \frac{\frac{[ {Ca}_{i} - {Ca}_{f} ]}{V o}}{C {a}_{f}} \qquad Eq . l$$

where:
Kd: oil-water partition coefficient of the partitioning tracer;
Cₒ: concentration of the partitioning tracer in the oil phase;
Cₐ: concentration of the partitioning tracer in the aqueous phase;
Cₐᵢ: concentration of the partitioning tracer in the aqueous phase before water-oil equilibrium;
C_{af}: concentration of the partitioning tracer in the aqueous phase after water-oil equilibrium; and
Vₒ: volume of the oil phase.

### Calculation of Sorm by Volumetric Balance

The calculation of Sorm by volumetric balance (step a₇) is performed using Equation 2.

$$S o r = \frac{[ {V}_{p} \cdot ( 1 - {S}_{w i} ) ] - [ ( {V}_{o m} - {V}_{g i} \cdot {v}_{c} ) \cdot {B}_{o} - {V}_{m} ]}{V p} \qquad Eq . 2$$

where:
Vₚ: Porous volume;
S_{wi}: Irreducible water saturation;
Vₒₘ: Volume of oil produced under atmospheric conditions (dead oil);
V_{gi}: Volume of gas injected under reservoir conditions (at test pressure and temperature);
V_{c}: Volume of condensed gas under atmospheric conditions/Volume of gas injected under reservoir conditions;
Bₒ: Formation volume factor=volume of oil in reservoir conditions (live oil)/volume of oil in atmospheric conditions (dead oil); and
Vₘ: output dead volume.

### Calculation Through Tracer Injection

The calculation of Sorm from tracer injection can be performed either analytically or numerically.

The analytical interpretation is based on the time lag between the partitioning and non-partitioning tracer profiles (normalized tracer concentration×porous volumes produced). Equations 3 to 5 are applied at specific points of the tracer elution profiles, and an average of the values found at each point is calculated to arrive at the final value.

$$S o r = \frac{{β}_{i}}{( {β}_{i} + {K}_{d} )} \qquad Eq . 3$$

$${β}_{i} = ( \frac{{v}_{water}}{{v}_{partitioning tracer}} ) - 1 or {β}_{i} = ( \frac{{V}_{partitioning tracer}}{{V}_{water}} ) - 1 \qquad Eq . 4 and 5$$

where:
βᵢ: delay factor of the partitioning tracer;
v_{water}: water velocity=velocity of the non-partitioning tracer;
v_{partitioning tracer}: velocity of the partitioning tracer;
v_{water}: elution volume of water=elution volume of the non-partitioning tracer;
v_{partitioning} tracer: elution volume of the partitioning tracer; and
Kᵢ=water-oil partition coefficient of the partitioning tracer

In numerical interpretation, a model of the experiment is assembled in a porous media flow simulator, which offers the possibility of injecting and considering several phenomena associated with the flow of tracers, such as water-oil partition; physical dispersion; molecular diffusion and adsorption.

Unlike analytical interpretation, numerical interpretation considers the complete elution profiles, and not just at certain points. It also allows taking into account some non-idealities and phenomena that cannot be fully captured in analytical interpretation, such as the attribution of different residual oil saturations to different layers, in heterogeneous samples and molecular diffusion.

Numerical interpretation begins with the adjustment of the elution profile of the non-partitioning tracer. It functions as a source of information for characterizing the pore space itself. The more heterogeneous the sample, the more layers of different permeabilities and thicknesses are required to adjust the entire non-partitioning tracer profile (shoulders, plateaus and rising/falling trends). The profile is usually adjusted by the number of layers and their hydraulic conductivities (thickness and absolute permeability). Once a good adjustment of the non-partitioning tracer has been achieved, the next step is to adjust the elution profile of the partitioning tracer, which is achieved simply by varying the residual oil saturation of each layer.

### Terms

The following nomenclatures were used in the present invention:

Bₒ: Oil formation volume factor. It is the ratio between the volume of oil in reservoir condition (live oil) and the volume of dead oil resulting from the depressurization/cooling of this oil to atmospheric condition.

Rich or wet gas: Gas rich in light petroleum components, condensable in ambient conditions.

Poor or dry gas: Gas without condensable components in ambient conditions.

WAG injection: Advanced oil recovery method where alternating cycles of water and gas (WAG-Water-Alternating-Gas) are injected.

Immiscible gas injection: This is where the gas-oil interfacial tension is high, generally above 1 dyne/cm. The system pressure is lower than the minimum miscibility pressure. Gas and oil remain as distinct phases, despite the transfer of mass between these phases, such as dissolution of gas in oil and absorption of light components of oil by the gas, up to a certain equilibrium limit.

Near miscible gas injection: This is where the gas-oil interfacial tension is close to zero (<1 dyne/cm), but not zero. The system pressure is very close to the minimum miscibility pressure but is still lower than it. Gas and oil also remain as distinct phases.

Miscible injection: This is where the interfacial tension is zero. The system pressure is higher than the minimum miscibility pressure. Gas and oil form a single phase.

MMP: Minimum miscibility pressure. For a given temperature and gas and oil compositions, this is the lowest pressure where the gas-oil interfacial tension nullified and where, consequently, the gas and oil phases cannot be distinguished.

Sorw: Residual oil saturation with continuous water injection.

Sorg: Residual oil saturation with continuous gas injection, usually immiscible or close to miscible condition.

Sorm: Residual oil saturation with gas injection, usually continuous, miscible.

### Examples

Those skilled in the art will value the knowledge shown herein and will be able to reproduce the invention in the embodiments indicated and in other variants, covered by the scope of the attached claims. Thus, the following example describes the apparatus, rock/fluid samples and experimental procedures used.

### Experimental Apparatus

The system used in this example is the one illustrated in FIG. 1.

### Rock Samples, Fluids and Tracers

The validation of the methodology was done using rock samples (represented in the tables below) and fluids from Brazilian fields.

The selected rock samples were grouped into composite plugs as described in Table 1. Composite plugs, which are longer and have greater porous volume, offer advantages in displacement tests, such as greater accuracy in determining pressure and produced volumes.

TABLE 1
Rock samples evaluated

| Rock | K | Phi | Swi |
|---|---|---|---|
| samples | (mD) | (%) | (%) |
| 1 | 45.5 | 14.8 | 26 |
| 2 | 99.1 | 15.3 | 21.6 |
| 3 | 111.5 | 15.3 | 23.7 |
| 4 | 513 | 18.7 | 26.2 |
| 5 | 1276.8 | 21.1 | 36.3 |
| 6 | 9.8 | 11.8 | 16.2 |
| 7 | 32 | 12.9 | 18.1 |
| 8 | 79.1 | 16.7 | 29.7 |
| 9 | 380.9 | 19.9 | 36.2 |
| 10 | 375.2 | 16.8 | 18.3 |
| 11 | 17.6 | 13.2 | 42.7 |
| 12 | 86.1 | 19.8 | 20.9 |
| 13 | 138.4 | 13.2 | 15 |
| 14 | 63.9 | 18 | 16.5 |
| 15 | 406.2 | 14.7 | 10.7 |

The tests were performed using recombined live oils. The gases used were hydrocarbon gases (C₁-C₄) containing varying levels of CO₂, in addition to pure CO₂. Seawater and synthetic formation water from each of these fields were also used.

The minimum miscibility pressures (MMP) for the different oils and gases used were previously determined in the laboratory using the rising bubble method.

The partitioning and non-partitioning tracers chosen were ethyl acetate and ethanol, respectively.

### Experiment Planning

The experiments in reservoir conditions were planned considering the following gas injections:
1) Injection in a condition close to miscible, wherein the injection was made with gas and oil pre-equilibrated with each other, until the mass transfer between the phases ceased, at a pressure of 100 psi (689.48 kPa), lower than the minimum miscibility pressure.
2) Injection in a miscible condition, with wet gas, wherein the pre-equilibration between the phases was made as in item (1) above, in order to enrich the gas with light components of the oil. However, the injection itself was made above the minimum miscibility pressure.
3) Injection in a miscible condition, with dry gas, wherein the injection was made above the minimum miscibility pressure, as in item (2) above, but the gas was not pre-equilibrated with the oil.
4) Injection in miscible condition, with dry gas, varying the CO₂ content, wherein the injection is identical to item (3) above, varying the CO₂ content in the gas.
5) WAG injection (alternating injection of water and gas) in miscible condition, with dry gas, wherein the injection is identical to item (3), alternating gas and water cycles (5% of the porous volume of the sample for each gas or water cycle).

### Procedure for Gas Injections

The procedure adopted consisted of the following steps:
Preparation of rock samples 1 to 15;
   Cleaning and determination of absolute permeability and effective porosity of the rock samples;
   Saturation with formation water and establishment of Swi;
Mobilization of the equipment;
   Confinement of the rock samples;
   Preparation of vessels with live oil;
   Pressurization and heating;
   Leak test;
Restoration of wettability;
Pre-equilibrium between phases, at a pressure 100 psi (689.48 kPa) lower than the minimum miscibility pressure;
Determination of Sorg by continuous injection of gas in a condition close to miscible;
Pore pressure elevation to a value higher than the minimum miscibility pressure;
Determination of Sorg by continuous injection of wet gas in miscible condition;
Determination of Sorg by continuous injection of dry gas in miscible condition (Sorm);
Injection of deionized water;
Injection of partitioning and non-partitioning tracers, at a concentration of 1% v/v in deionized water;
Collection of produced water samples at intervals of 5 porous volumes, for quantification of the tracers.

Between gas injections, to reabsorb it and return the sample to the initial condition of So=1−Swi, an injection of live oil was performed, until the relative permeability to oil (Kro@Swi) returned to its original value.

Some samples were demobilized; cleaned and again subjected to the entire previous preparation process, followed by one of the following steps:
Determination of Sorg by continuous injection of dry gas in miscible condition (Sorm), varying the CO₂ content of the gas; or
Determination of Sorg by alternating injection of miscible dry gas and water (cycles of 5% of the porous volume).

### Determination of the Water-Oil Partition Coefficient

The determination of the partition coefficient of the partitioning tracer was made with the fluids and temperature used in the tests, through the method known as bottle test. In this method, a certain volume of aqueous solution with a known concentration of partitioning tracer is placed in a bottle with a known volume of dead oil. This bottle is heated and stirred periodically for 24 hours to promote water-oil equilibrium, at the end of which time the concentration of tracer in the aqueous phase is sampled and quantified. The partition coefficient was calculated using Equation 1, as described in the Sorm calculation section.

The value found for the ethyl acetate partition coefficient in the oils evaluated was very similar in both cases, with a value of 4.73 being adopted.

### Calculation Through Volumetric Balance

The calculation of Sorm by volumetric balance was performed using Equation 2, as described in the Sorm calculation section.

### Calculation Through Tracer Injection

The calculation of Sorm from the injection of tracers was performed both analytically and numerically (as described in detail in the Sorm calculation section). In short, the analytical interpretation was based on the time lag between the profiles of the partitioning and non-partitioning tracers, with Equations 3 to 5 being applied. In the numerical interpretation, the experiment model was assembled in a numerical reservoir simulator.

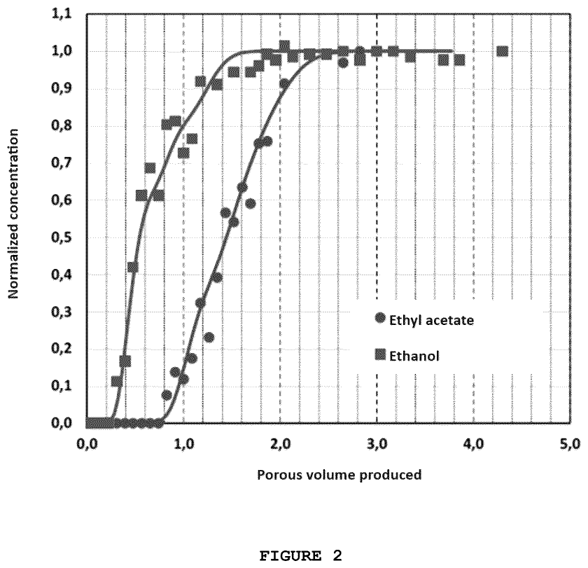

Regarding the numerical interpretation, FIG. 2 shows an example of an adjustment of the production profiles of the partitioning and non-partitioning tracers for sample 5.

### Results and Discussions

The main results and discussions to be highlighted were grouped as follows:
Sorm results;
Effect of miscibility/extraction mechanisms;
Effect of CO₂ content in the injected gas; and
Effect of gas injection method.

### Sorm Results

Table 2 shows the results of residual oil saturation for continuous injection of dry miscible gas, quantified by volumetric balance and injection of tracers in the evaluated samples. In all cases, however, the miscible condition was respected, with injections above the MMP.

In general, there was excellent congruence between both methods, which reduces the uncertainty regarding the measured values. Exceptions to this rule, however, were observed in two samples (Rock 6 and Rock 9). In these cases, it was assumed that the Sorm indicated by the tracer was abnormally low, with the correct value being that determined by the volumetric balance.

These are the values to be used as benchmarks for reservoir simulations, after the appropriate upscaling treatments, and are considered the lowest residual oil saturations that can be obtained with continuous gas injection.

TABLE 2
Sorms measured in evaluated samples

| | | | | | Sorm | Sorm |
|---|---|---|---|---|---|---|
| | Rock | K | Phi | Swi | BV¹ | Trace² |
| | samples | (mD) | (%) | (%) | (%) | (%) |
| | 1 | 45.5 | 14.8 | 26 | 21.2 | — |
| | 2 | 99.1 | 15.3 | 21.6 | 7.9 | — |
| | 3 | 111.5 | 15.3 | 23.7 | 12.9 | 13.8 |
| | 4 | 513 | 18.7 | 26.2 | 14.4 | 12.9 |
| | 5 | 1276.8 | 21.1 | 36.3 | 10.5 | 15.4 |
| Average of Rocks 1 to 5 | | | | | 13.40 | 14.0 |
| | 6 | 9.8 | 11.8 | 16.2 | 24.1 | 6.4* |
| | 7 | 32 | 12.9 | 18.1 | 17.3 | 15.1 |
| | 8 | 79.1 | 16.7 | 29.7 | 13.7 | 5.8 |
| | 9 | 380.9 | 19.9 | 36.2 | 18.5 | 17.2* |
| Average of Rocks 6 to 8 | | | | | 18.4 | 16.2 |
| | 10 | 375.2 | 16.8 | 18.3 | 2.4 | — |
| | 11 | 17.6 | 13.2 | 42.7 | 6.5 | 6.1 |
| | 12 | 86.1 | 19.8 | 20.9 | 2.7 | — |
| Average of Rocks 10 to 12 | | | | | 3.9 | 6.1 |
| | 13 | 138.4 | 13.2 | 15 | 6.3 | — |
| | 14 | 63.9 | 18 | 16.5 | 5.7 | 7.0 |
| | 15 | 406.2 | 14.7 | 10.7 | 2.8 | 3.5 |

| | | |
|---|---|---|
| Average of Rocks 13 to 15 | 4.9 | 5.2 |

¹Sorm obtained by volumetric balance
²Sorm obtained by tracers
*Value disregarded in the average calculation

### Effects of Miscibility and Extraction

While in water injection the relevant recovery mechanism is oil displacement, in gas injection there are other specific mechanisms, namely: swelling and reduction of viscosity of the oil phase, (nullification miscibility of gas-oil interfacial tension) and extraction of light components from the oil phase by the percolating gas. All these additional effects are due to the greater physical-chemical similarity between these phases at reservoir pressures, something that does not exist in the water-oil system.

Therefore, much more than in the water-oil system, the absolute value of the residual oil saturation in the gas can vary depending on the conditions in which the injection is made, especially in terms of miscibility and the light extraction capacity shown by the gas.

The degree of approximation of miscibility influences the porous fraction that can be accessed by the gas. The closer the gas-oil system is to the miscible condition, the lower the capillary forces and, consequently, the smaller the pores that can be penetrated by the gas, increasing the probability of contact and oil removal.

The gas extraction capacity can be translated by its potential to absorb components of lower molecular weight in the oil. This mass transfer process is determined not only by the pressure and temperature conditions, but also by the content of pre-existing light components in the injected gas. A gas said to be “dry” or “poor”, with few dissolved light components, has a greater extraction capacity than a gas said to be “rich” or “wet”.

Table 3 compares the Sor results measured with the continuous injection of approximately 5 porous volumes of gas in the following three conditions:
Near miscible condition (PM). This condition is characterized by gas-oil interfacial tension (G/O IFT) lower than 1 dyne/cm, but not zero, and with gas and oil balanced between themselves. In this context, the mechanisms of gas-oil miscibility, mass transfer between these phases (extraction), swelling and reduction of oil viscosity are suppressed. From this perspective, it is a condition that approximates the Sor measured in conventional special petrophysical analyses, whether in the water-oil or immiscible gas-oil system, where the phases, as a rule, are balanced between themselves prior to injection. The capacity of the gas to displace the oil is assessed, but the other recovery mechanisms typical of gas injection are not observed.
· Miscible condition, with rich gas. Rich gas is obtained by contact of dry gas with oil, prior to injection, so that it contains these components a priori. The injection is carried out, however, at a pressure and temperature where there is miscible behavior. In this way, the extraction recovery mechanism is limited, while the miscibility mechanism remains present.
Miscible condition, with poor gas. In this condition, the particular mechanisms of gas injection are fully present, leading to the lowest oil saturation value that can be obtained.

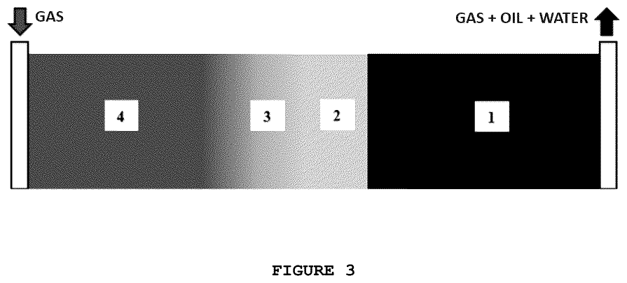

FIG. 3 illustrates the representativeness of the Sor determined in each of these conditions, for different portions of a reservoir subjected to miscible gas injection, where the numerical indices describe the following regions:
Index 1 indicates the region after the miscible zone, with original oil saturation;
Index 2 indicates the region corresponding to the miscible zone, where miscibility is achieved, but the mass transfer effects are restricted because the gas has already been enriched with light oil components on the way from the injection well to the miscible front region. The representative Sorm would be that obtained with the injection of rich miscible gas;
Index 3 indicates the region just after the miscible zone, where miscibility with residual oil has already been lost, but the gas-oil interfacial tension (IFT G/O) is very close to zero and the gas is rich in light oil components. The representative Sor would be that obtained with the injection of gas in a condition close to miscible; and
Index 4 indicates the region before the miscible zone, where the gas-oil mass transfer effects occur extensively because the injected gas does not contain light oil components. The representative Sorm would be that obtained with the injection of dry miscible gas. It is the lowest Sor value that can be obtained in a portion of rock widely swept by the continuously injected miscible gas.

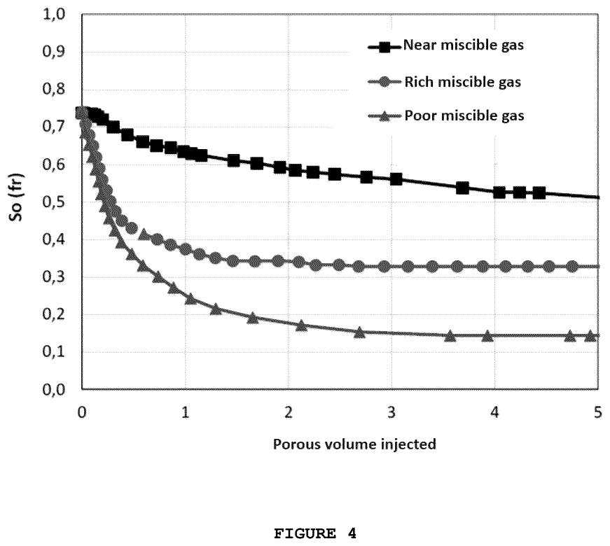

The results show how the Sor determined in conditions close to miscible and pre-equilibrated gas-oil, where additional gas recovery mechanisms are suppressed, is significantly higher than that obtained as they are activated, in the injection of wet miscible gas or dry miscible gas. FIG. 4 illustrates this difference for one of the samples (Rock 4) analyzed.

The data, therefore, highlight the importance of measuring Sorm in the most representative condition possible of field injection, with dry gas.

TABLE 3
Sorms measured in different injection conditions
in rock samples from the evaluated fields.

| Rock | Sor | Sor | Sor |
|---|---|---|---|
| sample | PM¹ | MR² | MP³ |
| 1 | — | — | 21.2 |
| 2 | 39.0 | 26.00 | 7.9 |
| 3 | 51.2 | — | 12.9 |
| 4 | 50.8 | 32.8 | 14.4 |
| 5 | 22.9 | 11 | 10.5 |
| 6 | — | 27.1 | 24.1 |
| 7 | 32.3 | 33.7 | 17.3 |
| 8 | 23.6 | 25 | 13.7 |
| 9 | 26.4 | 27.4 | 18.5 |
| 10 | 45.1 | — | 2.3 |
| 11 | 31.9 | — | 5.9 |
| 12 | 48.5 | — | 3.5 |
| 13 | 31.4 | — | 8.7 |
| 14 | 43.9 | — | 4.1 |
| 15 | 44.3 | — | 2.6 |

¹Sor obtained in a condition close to miscible
²Sor obtained in a miscible condition, with rich (wet) gas
³Sor obtained in a miscible condition, with poor (dry) gas

### Effect of CO₂ Content in the Injected Gas

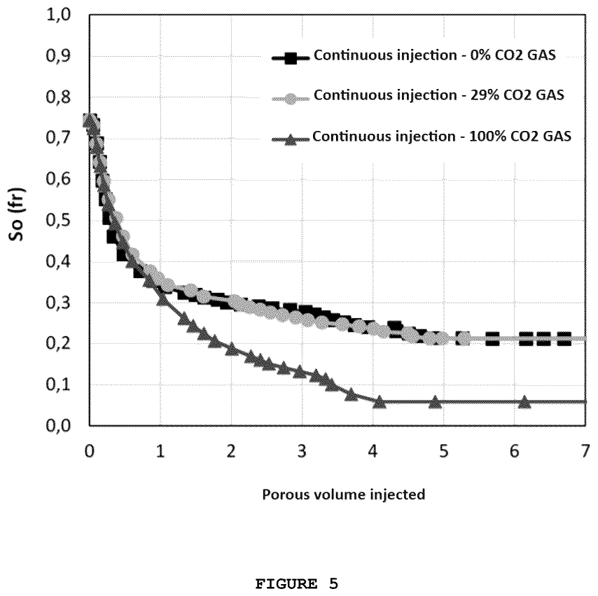

The experiments carried out with gases containing different CO₂ contents showed that this component only had an influence on Sorm, if its concentration was raised to very high levels (Table 4). In one of the samples evaluated (Rock 1), for example, the Sorm obtained with the injection of pure CO₂ was about 4 times lower than that obtained with hydrocarbon gas, free of CO₂ (FIG. 5). At low to intermediate contents the impact was insignificant. In other samples evaluated the observation was similar, although the already very low Sorm levels, even with low CO₂ contents, make it less evident.

Since all injections were made in miscible conditions, this result is attributed to the high solvency power of carbon dioxide compared to natural gas, which intensifies the extraction mechanism and highlights the value of injecting CO₂-rich streams for oil recovery, in typical CCUS (CO₂ Capture, Use and Geological Storage) applications.

TABLE 4
Sorms measured at different CO₂ contents
in samples from the evaluated fields

| | Sorm | Sorm | Sorm | Sorm |
|---|---|---|---|---|
| Rock | 0% | 29% | 60% | 100% |
| sample | CO₂ | CO₂ | CO₂ | CO₂ |
| 1 | 21.2 | 21.2 | — | 5.8 |
| 10 | — | 2.3 | 2.4 | 1.5 |
| 11 | — | 5.9 | 6.5 | — |
| 12 | — | 3.5 | 2.7 | — |
| 13 | — | 8.7 | 6.3 | 2.4 |
| 14 | — | 4.1 | 5.7 | — |
| 15 | — | 2.6 | 2.8 | — |

### Effect of Injection Method (Continuous×WAG)

Alternating water and gas (WAG) injection has the role of reducing gas permeability, controlling its mobility and increasing its sweep.

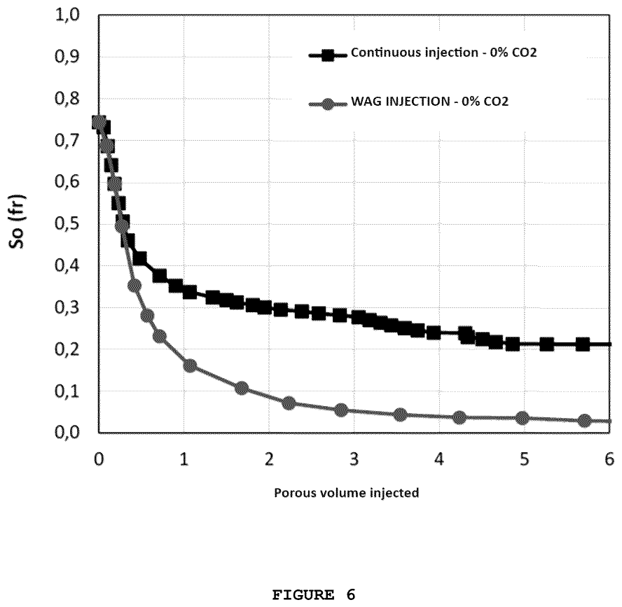

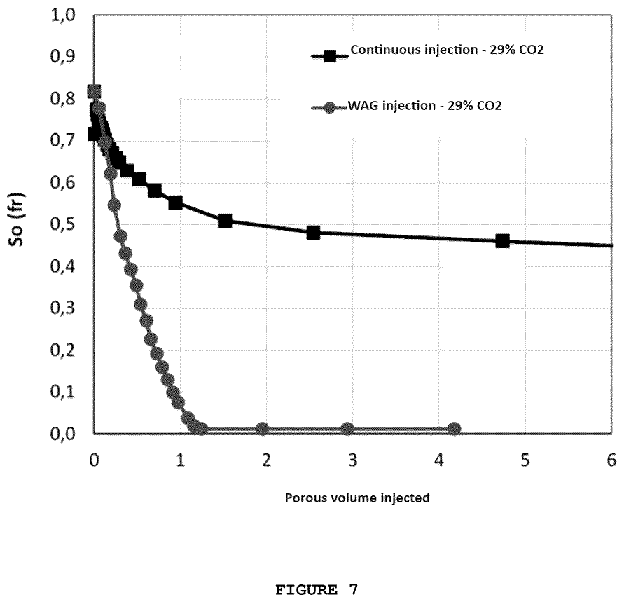

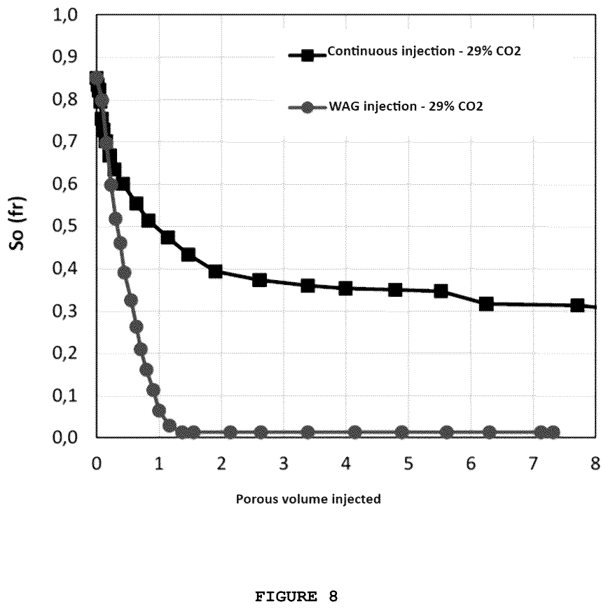

In the experiments in which the Sorm of continuous injection of miscible gas was compared with the Sorm of miscible WAG injection, the latter proved to be significantly lower (Table 5). Oil production was also anticipated (FIGS. 6 to 8). This demonstrates the potential of alternating injection for controlling gas mobility and increasing oil recovery, even in cases where the injected gas shows exacerbated miscibility and extraction effects, contacts the rock very well (plug experiment) and is rich in CO₂.

TABLE 5
Sorms measured in different injection methods (Continuous
× WAG) in samples from the fields evaluated

| Rock | Sorm | Sorm |
|---|---|---|
| sample | Continuous | WAG |
| 1 | 21.2 | 2.8 |
| 10 | 2.3 | 1.15 |
| 13 | 8.7 | 1.3 |

It was possible to quantitatively determine, using core samples and representative reservoir conditions, the residual oil saturation for miscible gas injection in different scenarios. The use of the Sorm quantification method by tracers, sequentially to the volumetric balance, allowed reducing the uncertainty in determining this parameter to levels as low as ±2 percentage points.

The results, after the appropriate upscaling and scaling treatments, can be used as benchmarks for reservoir simulations and profile interpretations, contributing to the reduction of uncertainties in reserve calculations and predictions of oil production curves; improving field management and increasing accuracy in the valuation and feasibility analysis of gas injection projects.

The developed methodology can also be applied to immiscible injections.

Therefore, the potential advantages of using more reliable values are: reduced uncertainty in reserve calculations and predictions of oil production curves, better field management, greater accuracy in the valuation and feasibility analysis of gas injection projects.

1- A method for determining the residual oil saturation by miscible gas injection comprising the sequential and independent in situ steps of:
(a) volumetric balance; and
(b) partitioning tracer injection.

2- A method according to claim 1, wherein the volumetric balance step comprises:
(a₁) filling the bypass of the sample holder and transducer lines with deaerated water;
(a₂) injecting live oil through the sample;
(a₃) filling the gas pumps with the test gas;
(a₄) injecting miscible gas with collecting the oil produced in atmospheric conditions (dead oil) over time, at a flow rate of the order of 0.5 to 1 cm³/min;
(a₅) determining the volume of condensate produced by the gas;
(a₆) determining the dead volume at the sample holder outlet by means of successive injections of dead water (deionized water) through the sample holder bypass and gas through the sample; and
(a₇) calculating the determination of Sorm.

3- A method, according to claim 1, wherein the step of partitioning tracer injection comprises:
(b₁) injecting deaerated water through the sample, after completion of the gas injection of step (a₄) of volumetric balance;
(b₂) injecting an aqueous solution containing 1% v/v of partitioning tracer and 1% v/v of non-partitioning tracer at a flow rate of 1 cm³/min, collecting samples under atmospheric conditions at each fraction of 5% of the porous volume;
(b₃) forwarding the collected samples for quantification of tracers, by means of gas chromatography and/or spectrophotometry in the infrared region; and
(b₄) calculating the determination of Sorm.

4- A method, according to claim 2, wherein the determination of the dead volume of step (a₆) optionally occurs through the inert gas expansion method.

5- A method, according to claim 3, wherein the partitioning tracer of step (b₂) is ethyl acetate.

6- A method, according to claim 3, wherein the non-partitioning tracer of step (b₂) is ethanol.

7- A method, according to claim 2, wherein the calculation of Sorm by volumetric balance (step a₇) is performed through the following equation:
$$Sor = \frac{[ {V}_{p} \cdot ( 1 - {S}_{w i} ) ] - [ ( {V}_{o m} - {V}_{g i} \cdot {v}_{c} ) \cdot {B}_{o} - {V}_{m} ]}{V p}$$

8- A method, according to claim 3, wherein the calculation of Sorm by injection of tracers is carried out both analytically and numerically.

9- A method, according to claim 8, wherein the analytical form calculates an average of the values found at each of the points of the elution profiles of the tracers by applying the following equations:
$$S o r = \frac{{β}_{i}}{( {β}_{i} + {K}_{d} )}$$
$${β}_{i} = ( \frac{{v}_{water}}{{v}_{partitioning tracer}} ) - 1 or {β}_{i} = ( \frac{{V}_{partitioning tracer}}{{V}_{water}} ) - 1 .$$

10- A method, according to claim 8, wherein the numerical form calculates through a flow simulator in porous media.
